(19) United States
(12) Patent Application Publication
(10) Pub. No.: US 2019/0167831 A1
(43) Pub. Date: Jun. 6, 2019

(54) SCENT ENHANCED TRAJECTORY

(71) Applicant: International Business Machines Corporation, Armonk, NY (US)

(72) Inventors: Yuk L. Chan, Rochester, NY (US); Michael D. Essenmacher, Danbury, CT (US); David B. Lection, Raleigh, NC (US); Eric L. Masselle, Wake Forest, NC (US)

(21) Appl. No.: 15/828,691

(22) Filed: Dec. 1, 2017

(51) Int. Cl. A61L 9/12; H04W 4/02; H04W 4/00

(52) U.S. Cl. CPC A61L 9/122; H04W 4/005; H04W 4/023; A61L 9/125

(57) ABSTRACT

Specified scents are generated having a strength corresponding to the proximity of the devices to a target object. Scent generation is adjustable according to pre-defined parameters associated with type of scent, location of an enabled device, weather conditions, geography, and the number of other enabled devices generating the type of scent. A virtual scent zone is generated by a scent-generating device according to a trajectory that provides for a user to move toward the target object by changing a characteristic of the generated scent according to the location of the scent-generating device along the trajectory.

### BACKGROUND

The present invention relates generally to the field of odor technology, and more particularly to selectively generated odors.

Perception of visual images, sounds, and smells includes a directional component. Peripheral vision is a good illustration of the directional component associated with visual images. Objects appearing just within one's peripheral vision may prompt a person to turn their head or direct their eyes toward the object. The directional components of hearing include a 360-degree detection of the sound source. Sounds are perceived in such a way that the sound may be measured for strength differential of the audio signals received at each ear. The perception of smell also has a directional component that allows a person to spatially locate a smell by simply turning their head in the direction of the source of smell.

Digital scent technology, also known as olfactory technology, involves generating olfactory representations. It is a technology that can include sensing, transmitting, and receiving digital media including: web pages, video games, movies and music. Sensing is performed with olfactometers and/or electronic noses.

### SUMMARY

In one aspect of the present invention, a computer-implemented method, a computer program product, and a system for distance-based scent emission includes: (i) transmitting, by a scent-generating device, first location information; (ii) receiving scent instructions based on the first location information, the scent instructions including an algorithm for emission of a specified scent; and (iii) determining a first scent emission response according to the scent instructions. The first location information includes a device location at the time of the transmitting action.

In another aspect of the present invention, a computer-implemented method, a computer program product, and a system includes: (i) determining a device location of a first scent enabled device; and (ii) responsive to the device location being within a pre-defined distance from a target location, causing the first scent enabled device to generate a scent having a characteristic being variable according to a distance between the device location and the target location. The characteristic is a measure of scent generated.

In yet another aspect of the present invention, a computer-implemented method, a computer program product, and a system includes: (i) receiving, by a scent-enabled device, a first scent instruction including a specified scent and a target location; (ii) determining a first distance from the target location based on a first location of the scent-enabled device; (iii) determining a first characteristic of the specified scent based on the first distance; and (iv) releasing the specified scent according to the determined first characteristic.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a flowchart showing a second embodiment method performed, at least in part, by the first embodiment system;

FIG. 4 is a flowchart showing a third embodiment method performed, at least in part, by the first embodiment system;

FIG. 5 is a block diagram view of a machine logic (e.g., software) portion of the first embodiment system;

FIG. 6 is a block diagram view of a fourth embodiment of a system according to the present invention;

FIG. 7 is a flowchart showing a fourth embodiment method performed, at least in part, by the fourth embodiment system;

FIG. 8 is a block diagram view of a machine logic (e.g., software) portion of the fourth embodiment system; and

FIG. 9 is an example diagram of users at varying distances from a broadcast transmitter.

### DETAILED DESCRIPTION

Specified scents are generated having a strength corresponding to the proximity of the devices to a target object. Scent generation is adjustable according to pre-defined parameters associated with type of scent, location of an enabled device, weather conditions, geography, and the number of other enabled devices generating the type of scent. A virtual scent zone is generated by a scent-generating device according to a trajectory that provides for a user to move toward the target object by changing a characteristic of the generated scent according to the location of the scent-generating device along the trajectory. This Detailed Description section is divided into the following sub-sections: (i) Hardware and Software Environment; (ii) Example Embodiment; (iii) Further Comments and/or Embodiments; and (iv) Definitions.

### I. Hardware and Software Environment

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

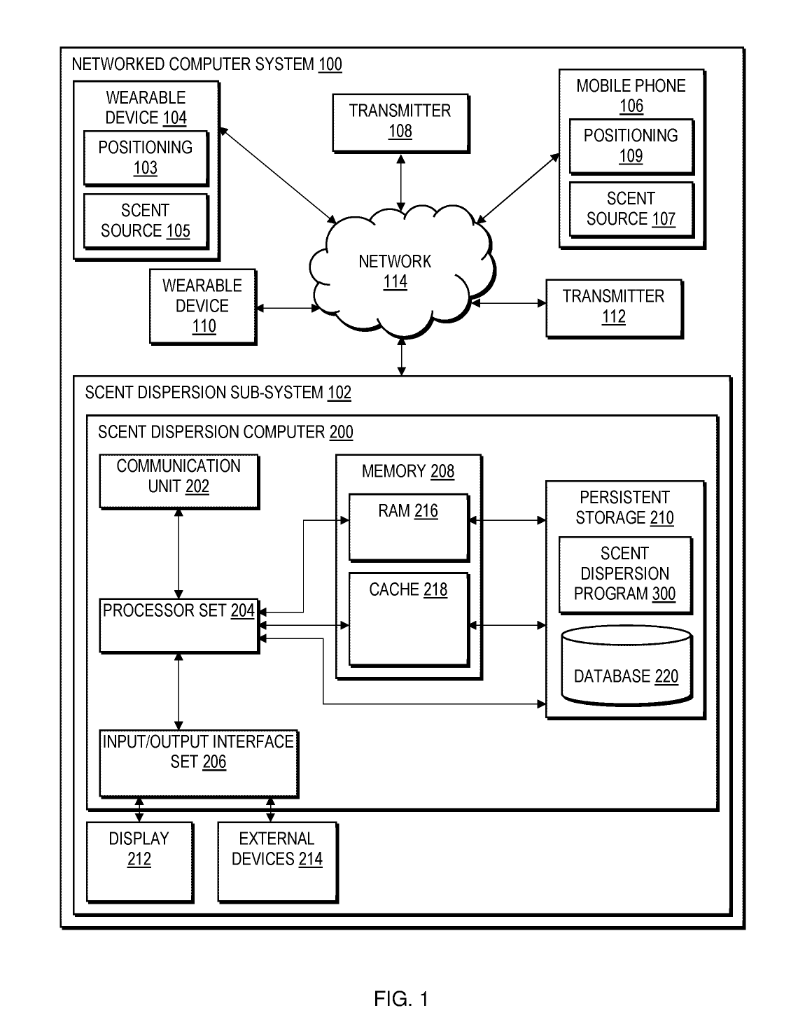

An embodiment of a possible hardware and software environment for software and/or methods according to the present invention will now be described in detail with reference to the Figures. FIG. 1 is a functional block diagram illustrating various portions of networked computers system 100, including: scent dispersion sub-system 102; wearable device 104, positioning module 103 (such as a global positioning system), scent source 105, mobile phone 106, positioning module 109 (such as a global positioning system), scent source 107, broadcast transmitter 108, wearable device 110, broadcast transmitter 112; and communication network 114. Scent dispersion sub-system 102 contains: scent dispersion computer 200; display device 212; and external devices 214. Scent dispersion computer 200 contains: communication unit 202; processor set 204; input/output (I/O) interface set 206; memory device 208; and persistent storage device 210. Memory device 208 contains: random access memory (RAM) devices 216; and cache memory device 218. Persistent storage device 210 contains: scent dispersion program 300 and database 220.

Scent dispersion sub-system 102 is, in many respects, representative of the various computer sub-systems in the present invention. Accordingly, several portions of scent dispersion sub-system 102 will now be discussed in the following paragraphs.

Scent dispersion sub-system 102 may be a laptop computer, a tablet computer, a netbook computer, a personal computer (PC), a desktop computer, a personal digital assistant (PDA), a smart phone, or any programmable electronic device capable of communicating with client sub-systems via communication network 114. Scent dispersion program 300 is a collection of machine readable instructions and/or data that is used to create, manage, and control certain software functions that will be discussed in detail, below, in the Example Embodiment sub-section of this Detailed Description section.

Scent dispersion sub-system 102 is capable of communicating with other computer sub-systems via communication network 114. Communication network 114 can be, for example, a local area network (LAN), a wide area network (WAN) such as the Internet, or a combination of the two, and can include wired, wireless, or fiber optic connections. In general, communication network 114 can be any combination of connections and protocols that will support communications between and client sub-systems.

Scent dispersion sub-system 102 is shown as a block diagram with many double arrows. These double arrows (no separate reference numerals) represent a communications fabric, which provides communications between various components of scent dispersion sub-system 102. This communications fabric can be implemented with any architecture designed for passing data and/or control information between processors (such as microprocessors, communications processors, and/or network processors, etc.), system memory, peripheral devices, and any other hardware components within a system. For example, the communications fabric can be implemented, at least in part, with one or more buses.

Memory device 208 and persistent storage device 210 are computer readable storage media. In general, memory device 208 can include any suitable volatile or non-volatile computer readable storage media. It is further noted that, now and/or in the near future: (i) external devices 214 may be able to supply some, or all, memory for scent dispersion sub-system 102; and/or (ii) devices external to scent dispersion sub-system 102 may be able to provide memory for scent dispersion sub-system 102.

Scent dispersion program 300 is stored in persistent storage device 210 for access and/or execution by one or more processors of processor set 204, usually through memory device 208. Persistent storage device 210: (i) is at least more persistent than a signal in transit; (ii) stores the program (including its soft logic and/or data) on a tangible medium (such as magnetic or optical domains); and (iii) is substantially less persistent than permanent storage. Alternatively, data storage may be more persistent and/or permanent than the type of storage provided by persistent storage device 210.

Scent dispersion program 300 may include both substantive data (that is, the type of data stored in a database) and/or machine readable and performable instructions. In this particular embodiment (i.e., FIG. 1), persistent storage device 210 includes a magnetic hard disk drive. To name some possible variations, persistent storage device 210 may include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage device 210 may also be removable. For example, a removable hard drive may be used for persistent storage device 210. Other examples include optical and magnetic disks, thumb drives, and smart cards that are inserted into a drive for transfer onto another computer readable storage medium that is also part of persistent storage device 210.

Communication unit 202, in these examples, provides for communications with other data processing systems or devices external to scent dispersion sub-system 102. In these examples, communication unit 202 includes one or more network interface cards. Communication unit 202 may provide communications through the use of either or both physical and wireless communications links. Any software modules discussed herein may be downloaded to a persistent storage device (such as persistent storage device 210) through a communications unit (such as communication unit 202).

I/O interface set 206 allows for input and output of data with other devices that may be connected locally in data communication with scent dispersion computer 200. For example, I/O interface set 206 provides a connection to external devices 214. External devices 214 will typically include devices, such as a keyboard, a keypad, a touch screen, and/or some other suitable input device. External devices 214 can also include portable computer readable storage media, such as, for example, thumb drives, portable optical or magnetic disks, and memory cards. Software and data used to practice embodiments of the present invention (e.g., scent dispersion program 300) can be stored on such portable computer readable storage media. In these embodiments, the relevant software may (or may not) be loaded, in whole or in part, onto persistent storage device 210 via I/O interface set 206. I/O interface set 206 also connects in data communication with display device 212.

Display device 212 provides a mechanism to display data to a user and may be, for example, a computer monitor or a smart phone display screen.

The programs described herein are identified based upon the application for which they are implemented in a specific embodiment of the invention. However, it should be appreciated that any particular program nomenclature herein is used merely for convenience, and thus, the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

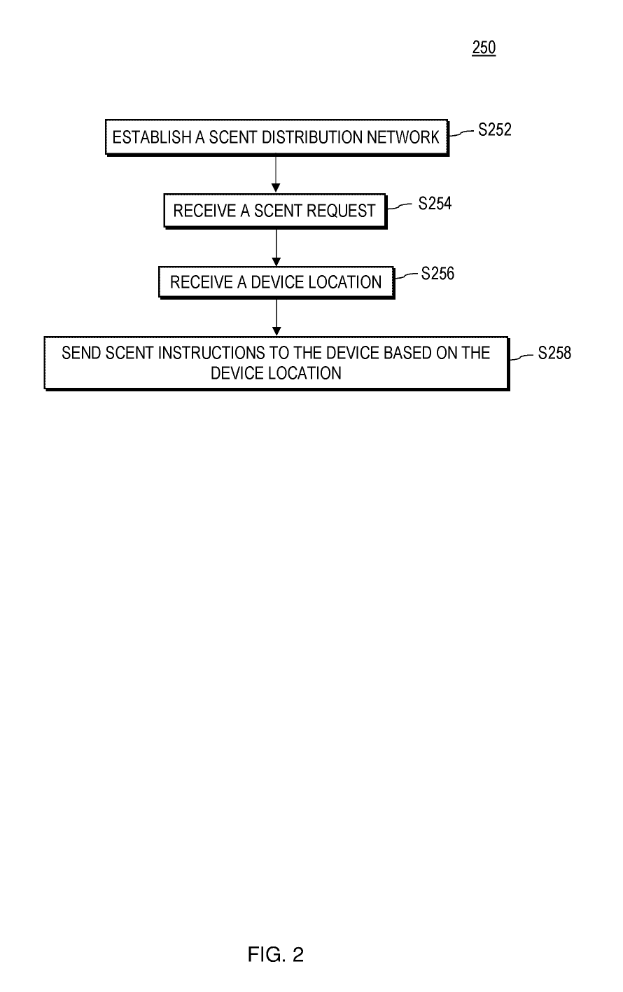

FIG. 2 shows flowchart 250 depicting a computer-implemented method according to an embodiment of the present invention.

Processing begins at step S252 where a scent distribution network is established. A scent distribution network may include a set of one or more mobile devices for emitting a scent, a scent manager, and a requesting user specifying a particular scent and a target location. With reference to FIG. 1, the set of one or more mobile devices includes, for example, wearable device 104 and 110 and mobile phone 106, the scent manager is scent dispersion sub-system 102, and the requesting user may be, for example, transmitters 108 and 112, where the transmitters are used to transmit requests to the scent manager. In this example, the scent distribution network operates to coordinate targeted scent generation according to transmitted target locations and in-network mobile device locations.

Processing proceeds to step S254, where the scent manager receives a scent request. In this example, the scent request includes a target location and a specified scent. Further in this example, the specified scent is a pre-defined scent selected from a set of scents. Alternatively, the specified scent is determined from a scent detecting device and components for making up the detected scent (such as a scent recipe) are transmitted to the scent manager via the scent request. In this example, the target location is tied to a stationary physical structure, such as a building where a restaurant is located or a recreational facility. Alternatively, the target location is virtual target object not corresponding to a physical object or structure. Alternatively, the target location is associated with a mobile object such that the target location may be updated over time according to the physical location of the mobile object.

Processing proceeds to step S256, where the scent manager receives a device location. The device location is a GPS location or other location coordinate associated with the location of the device sending the location to the scent manager. In this example, the device location is the GPS location determined by a mobile phone and submitted to the scent manager for processing. Alternatively, the device location is the location coordinates for a device within the established scent distribution network. According to some embodiments of the present invention scent manager requests the device location periodically. According to some embodiments of the present invention, the mobile device sends its GPS location for every 1000 feet of movement. Alternatively, the device location is received at a pre-determined time of day according to a pre-defined schedule.

Some embodiments of the present invention are directed to device locations that are virtual and/or target locations that are virtual. According to some fully virtual embodiments of the present invention, a user is immersed in a virtual environment. The target location corresponds to a virtual location within the virtual environment and the device location corresponds to a virtual location of the user.

Processing ends at step S258, where the scent manager sends scent instructions to the device based on the received device location. In this example, the scent request further includes a pre-defined distance from the target location. The pre-defined distance allows the scent manager to determine when to send scent instructions to a device. Alternatively, a pre-defined distance from a target location is per-determined by policy and is not established by a scent request. The scent instructions provide the device with information about release of a particular scent and may include: scent strength gradient, maximum distance from target location to release the scent, components making up the scent, and/or frequency for submitting the device location. The scent strength gradient refers to a defined strength of scent to be released according to a calculated, or otherwise determined, distance of the device from the target location.

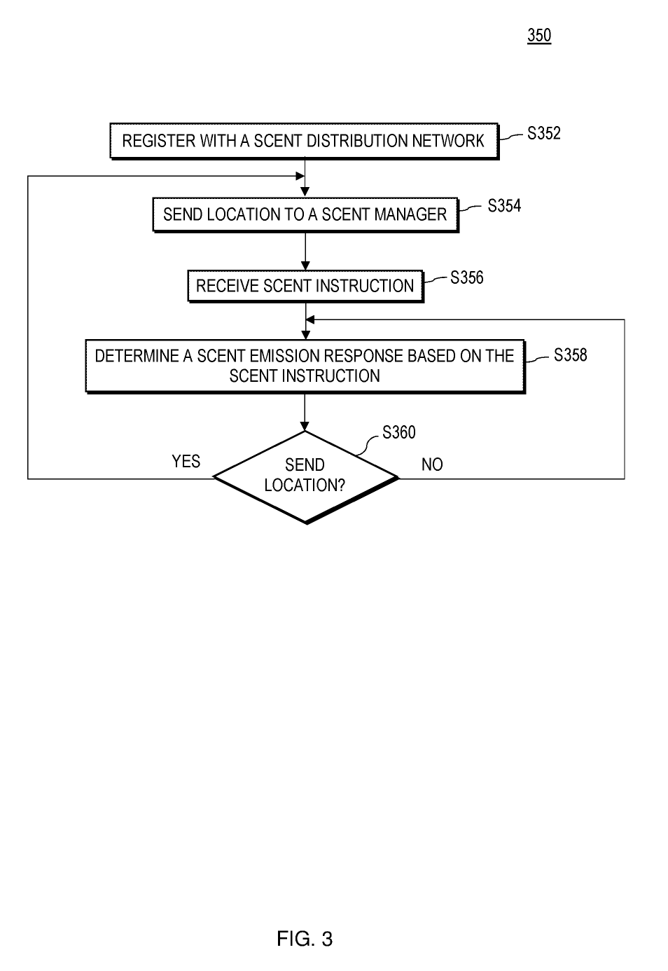

FIG. 3 shows flowchart 350 depicting a computer-implemented method according to an embodiment of the present invention.

Processing begins at step S352, where a scent enabled device is registered with a scent distribution network. The scent enabled device produces or releases scents. In this example, the scent enabled device is a mobile phone having a variety of pre-defined scents for releasing according to scent instructions. Alternatively, the scent enabled device has access to a variety of scent components for combining according to scent instructions to produce a particular customized scent. In this example, the registration authorizes the scent manage, responsive to receiving a device location, to send scent instructions to the device for automatic scent emission response. Alternatively, the authorization is only for scent instructions to be received, but are followed according to user-input. Some embodiments of the present invention are directed to a registration that authorizes the scent manager to detect when the scent-enabled device is within a pre-defined distance from a target location and to automatically send the scent instructions to the scent-enabled device.

Processing proceeds to step S354, where the scent-enabled device sends a device location to the scent manager. The device location is a GPS location or other location coordinate associated with the location of the device sending the location to the scent manager. In this example, the device location sent by the scent-enabled device corresponds to a physical location of the scent-enabled device. Alternatively, the device location corresponds to a virtual location of the scent-enabled device.

Processing proceeds to step S356, where the scent-enabled device receives a scent instruction. In this example, the scent instruction is received by the scent-enabled device in response to the device sending the device location. Alternatively, scent instructions are received based on a device location detected by the scent manager. Alternatively, multiple scent instructions are received by the scent-enabled device for various target locations and the scent-enabled device determines when a particular target location is within a pre-defined distance of the location of the scent-enabled device.

Some embodiments of the present invention are directed to providing to the scent-enabled device a set of scent instructions sufficient for autonomous processing with no further communication with the scent manager. Scent instructions may include a scent strength gradient that considers orientation of the scent enabled device. That is, when the scent-enabled device faces one direction, the strength of scent released may vary from the strength of scent released if the scent-enabled device were facing another direction. In that way, directional aspects of the scent instructions are tuned for an individual user. Slight directional changes may be further appreciated when applied to a virtual target location and virtual device location such that by turning the head, the scent release varies.

Processing proceeds to step S358, where the scent-enabled device determines a scent emission response based on the scent instructions. Scent emission responses may include notifying a user that a scent instruction is active (or that a scent is ready to release), automatically releasing a requested scent according to a requested scent strength gradient, and/or producing the customized scent according to specified scent components. In this example, scent emission responses are defined by the scent instruction. Alternatively, the scent emission response is defined by policy and not related to a particular scent instruction. For example, the scent-enabled device has only one scent to release, so upon receipt of the scent instruction, the scent-enabled device, by policy, determines a distance from the target location and releases the scent according a scent strength gradient governed by the policy. Some embodiments of the present invention determine a distance by calculating a shortest distance from the target location. Alternatively, the distance is calculated based on an available route for a user to take, whether walking, driving, and bicycling, etc. The available route may be made up of various segments and the distance is calculated by adding lengths of the various segments together.

Some embodiments of the present invention are directed to a scent-enabled device storing multiple scent instructions for use over a particular period of time. In that way, when a scent-enabled device is within range of one of the target locations, the scent-enabled device releases a scent according to the corresponding scent instructions. The release may be automatic or a user may be prompted to cause the scent-enabled device to release the scent. Regardless of the way in which the scent is released, it is released according to the corresponding scent instructions.

Processing proceeds to decision step S360, where the scent-enabled device determines whether or not to send the device location to the scent manager. Some embodiments of the present invention occasionally send device location information to the scent manager. Some embodiments of the present invention reconsider the received scent instructions periodically so a scent is released according to a recently computed scent strength gradient. Determination of when to resend device locations or to reconsider the scent emission response will vary by personal preference, scent distribution network policy, and/or the active scent instructions.

### III. Further Comments and/or Embodiments

Some embodiments of scent dispersion program 400 operate to cause certain connected devices to generate specified scents having a strength corresponding to the proximity of the devices to a target location. Scent generation is adjustable according to pre-defined parameters associated with location of the device relative to the target location. In some embodiments of the present invention, the target location corresponds to the location of a static physical object, such as a restaurant. Alternatively, the target location corresponds to a mobile location of a physical object, such as a food truck. In some embodiments of the present invention, scent generation is governed by pre-defined parameters associated with weather conditions and/or geography.

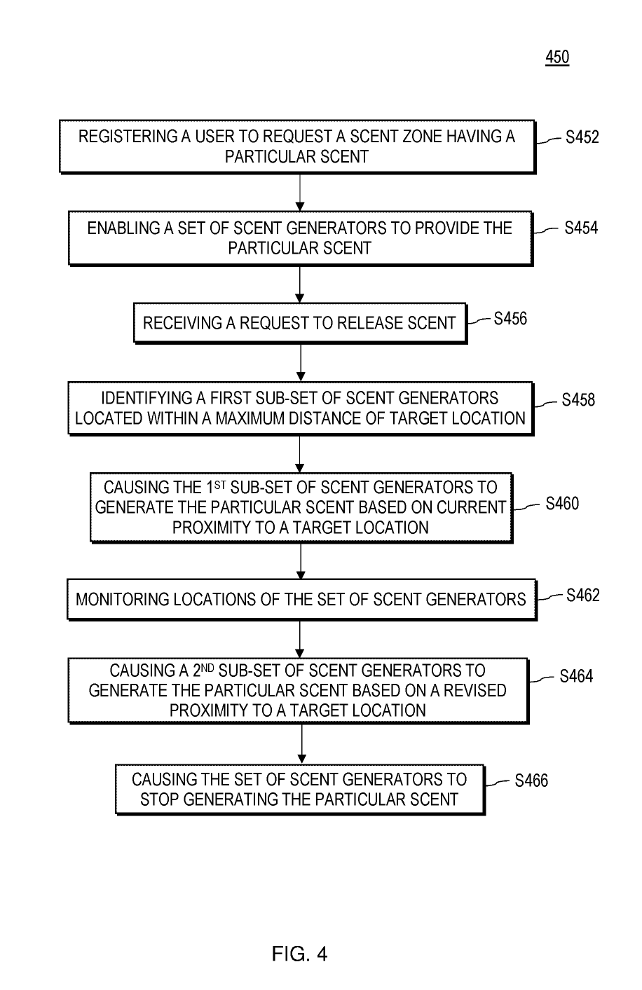

FIG. 4 shows flowchart 450 depicting a method according to the present invention.

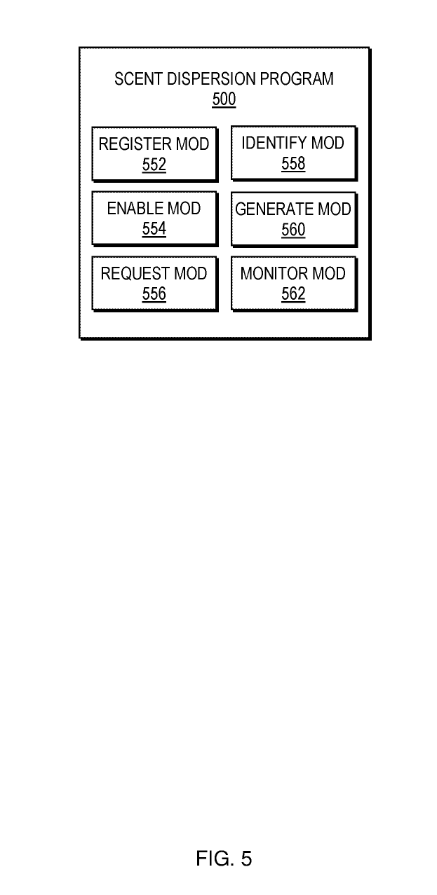

FIG. 5 shows scent dispersion program 500, which performs at least some of the method operations of flowchart 450. This method and associated software will now be discussed, over the course of the following paragraphs, with extensive reference to FIG. 4 (for the method operation blocks) and FIG. 5 (for the software blocks).

Processing begins at step S452, where register module (“mod”) 552 registers a user to request a scent guide be generated having a particular scent. In this example, registering provides for the user to disclose the scent profile(s) for use when sending scent instructions. Alternatively, digital scent technology is employed to analyze a scent provided by the user during the registration step. Alternatively, the user selects during registration a pre-defined scent(s) to be used for a scent release. By registering to request a scent, the user is in a position to have scent dispersion sub-system 102 send scent instructions to nearby enabled devices. In this example the user requests that scent instructions be released, but does not directly cause the scent generators to release the particular scent. Alternatively, scent instructions are prepared for the registered user upon registration and those instructions are set to expire at a pre-defined date or time.

Processing proceeds to step S454, where enable mod 554 enables a set of scent generators to produce a particular scent(s). Some embodiments of the present invention enable a single scent generator for use by a single individual for guiding the individual to the target location. Some embodiments of the present invention enable more than one scent generator such that multiple individuals are guided to the target location based on their various locations. In this example, a group of wearable devices are set up for discharging the particular scent(s). One example of a wearable device is shown in FIG. 1 as wearable device 104 with scent source 105 being set up to discharge the particular scent. Specifically, a scent dispersing unit is installed into the wearable device, such that automatic scent dispersion of the particular scent occurs upon receipt of input from scent dispersion sub-system 102 over network 114. Alternatively, the scent dispersing unit is initiated remotely and programmed to disperse the particular scent for the registered user.

Some embodiments of the present invention rely upon digital scent technology stored on the enabled device to release a requested scent. The digital scent technology is equipped to mix various scent components to produce the requested scent. In this case, the formula for creating a particular scent is transmitted to the enable device for dispersing on demand. Alternatively, the pre-defined scents are already loaded onto the enabled device for production when a registered user requests the scent.

Processing proceeds to step S456, where request mod 556 receives a request by a registered user to establish a scent guide. In this example, the request includes the scent type, the target location, the maximum distance away from the target location for scent generation, and the duration of scent generation. Alternatively, only the scent type is provided in a request where the registered user operates a transmitter on site, such as 108 and 112 in FIG. 1, at the target location, the distance from the target location and the duration are pre-defined. Alternatively, only one scent for a specified duration is registered by the user, so a request only includes the target location and the distance from the target location. Alternatively, the registration of the user includes a request having a start condition and an end condition. For example, upon registration, the user establishes that the submitted request will be effective in 3 days and will remain in effect for 5 days.

Processing proceeds to step S458, where identify mod 558 identifies a first sub-set of enabled scent generators located within the maximum distance from the target location. In some embodiments of the present invention, the distance information is provided with the request or upon registration of the user. In this example, scent generators such as wearable device 104 and mobile phone 106 have positioning modules 103 and 109 respectively. The position module is, in some embodiments of the present invention, a global positioning system (GPS). The identify mod determines locations of the various enabled scent generators by receiving inputs from the position systems. Those enabled devices within a pre-defined distance of the target location are identified as a first sub-set of scent generators.

Alternatively, scent generators are strategically located throughout a geographic area and identify mod 558 identifies those scent generators located within the pre-defined distance of the target location. According to this alternative, the scent generators are configured to release particular scents for nearby users to detect. For example, a neighborhood scent dispersion system is established that releases a certain intensity of a particular scent when an ice cream truck in on its route. The intensity of scent released varies according to the distance between the scent generator and the ice cream truck.

Some embodiments of the present invention reset the identified set of scent generators periodically according to the frequency provided in the scent request. Alternatively, the identified set is reset by default every quarter period of the specified duration of the scent guide. Resetting the identified set of scent generators is discussed in more detail with respect to step S464, below.

Processing proceeds to step S460, where generate mod 560 causes the first sub-set of scent generators to generate the requested scent based on a current proximity of the set of scent generators to the target location. It should be noted that individual use of scent generators is a likely scenario, that is, a single scent generator may be identified within a scent guide range for generating a particular scent having a strength, or other characteristic, based on the distance between the scent generator and the target location. The scent guide range establishes a maximum, and perhaps minimum, distance for releasing a particular requested scent, as a guide to the target location.

In this example, the scent is released for a varying period of time according to distance from target location. The closer the scent generator is to the target location, the longer the scent is released. Alternatively, a relatively stronger scent is released closer to the target location. Alternatively, the scent is generated in a relatively higher volume at locations closer to the target location. Essentially, a user wearing an enabled wearable device a greater distance from the target location smells a relatively light scent compared to what the user smells who is wearing an enable wearable device close to the target location.

Processing proceeds to step S462, where monitor mod 562 monitors locations of the set of enabled scent generators. During release of the requested scent, the monitor mod tracks the scent generators to identify a next set of scent generators for releasing additional scent. In this example, when one device, being relatively distant from the target location, moves closer to the target location, the monitor mod recognizes this change in location. Also, if a scent generator moves into the pre-defined distance from the target location, the monitor mod recognizes a new scent generator as being available for use. Alternatively, the target location is associated with a target object and the target object moves. Monitor mod 562 identifies enabled scent generators within the moving pre-defined distance from the target object.

Processing proceeds to step S464, where generate mod 560, causes a second sub-set of enabled scent generators to generate a particular scent as requested by the source and based on a revised proximity of the set of scent generators to the target location. In this example, the second sub-set of scent generators is identified by identify mod 558 after a pre-determined period of time and is based on observations reported by monitor mod 562. The second sub-set of enable scent generators are identified as being within the pre-defined distance from the target location. Individual locations of the scent generators are known and updated from the previously identified first set of scent generators. The specified scent is generated according to scent zone criteria based on individual proximity to the target location.

Processing ends with step S466, where generate mod 560 causes the set of enabled scent generators to stop generating the particular scent requested by the user. In this example, the release of the particular scent is performed for the requested duration. During the requested duration, identify mod 558 identifies new sub-sets of enabled devices halfway through the specified duration.

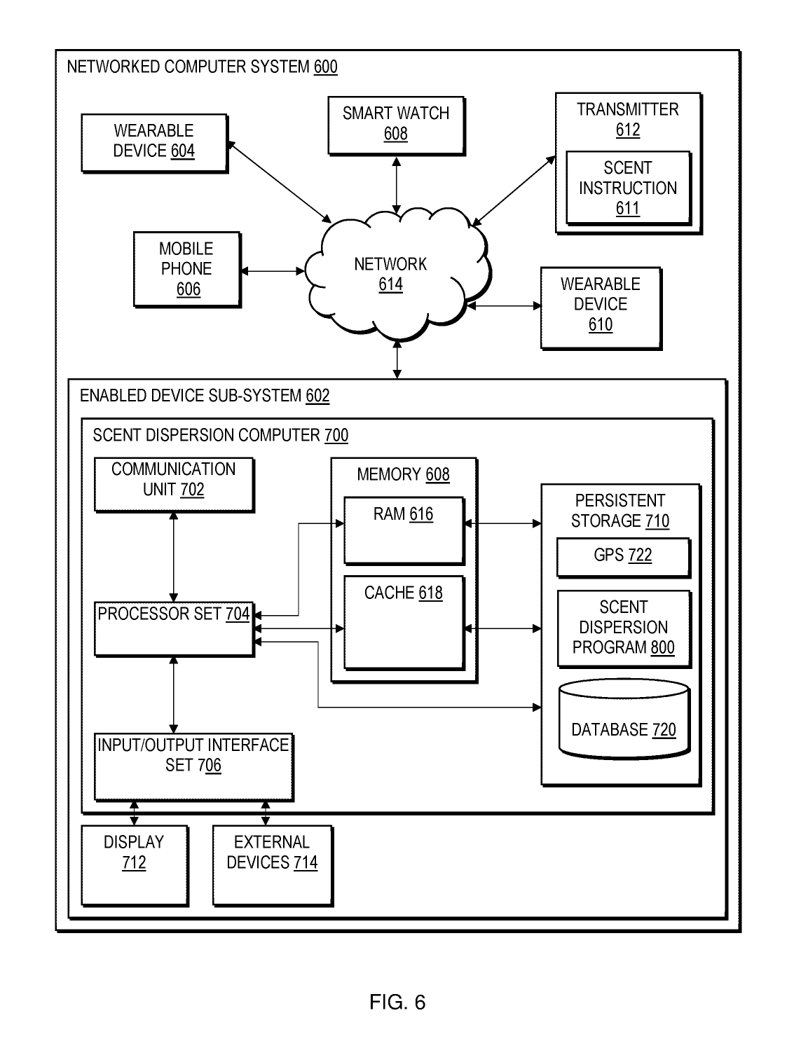

Another embodiment of a possible hardware and software environment for software and/or methods according to the present invention will now be described in detail with reference to FIGS. 6-9. FIG. 6 is a functional block diagram illustrating various portions of networked computers system 600, including: enabled device sub-system 602; wearable device 604, mobile phone 606, smart watch 608, wearable device 610, broadcast transmitter 612; and communication network 614. Scent dispersion sub-system 602 contains: scent dispersion computer 700; display device 712; and scent generator 714. Scent dispersion computer 700 contains: communication unit 702; processor set 704; input/output (I/O) interface set 706; memory device 708; and persistent storage device 710. Memory device 708 contains: random access memory (RAM) devices 716; and cache memory device 718. Persistent storage device 710 contains: database 720, global positioning system (GPS) 722, and scent dispersion program 800. System 600 is similar to system 100 (FIG. 1) and the discussion of the various elements of system 100 corresponds also to system 600, where applicable. Certain variations between the two systems are shown according to the specifically described functions associated with flowcharts 450 and 750.

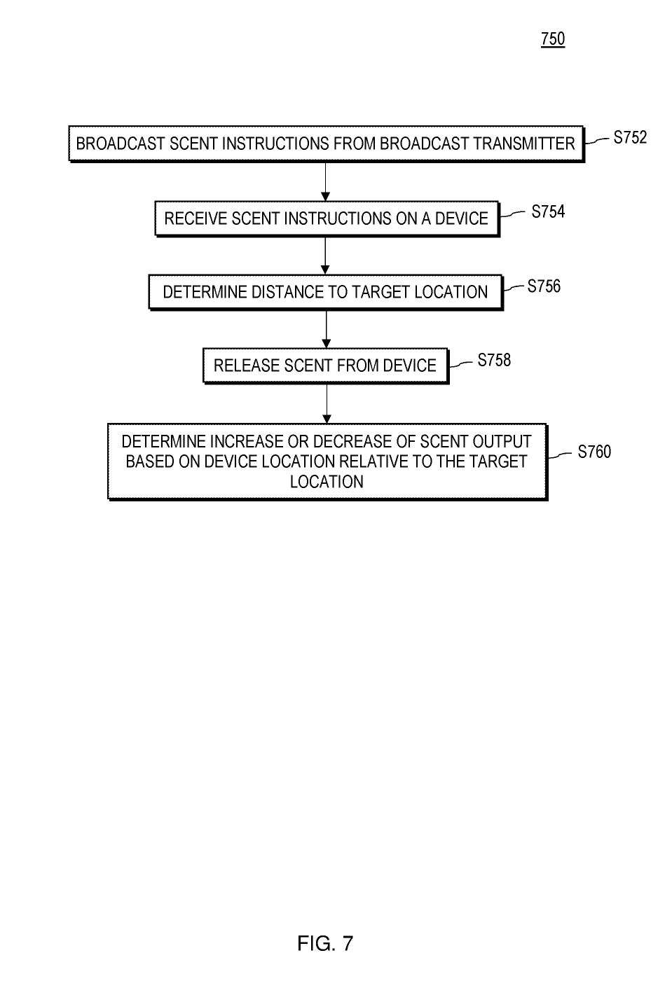

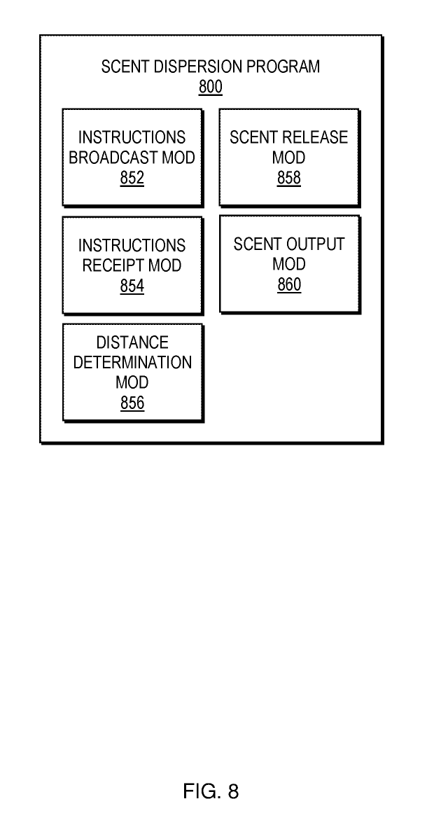

FIG. 7 shows flowchart 750 depicting a method according to the present invention. FIG. 8 shows scent dispersion program 800, which performs at least some of the method operations of flowchart 750. This method and associated software will now be discussed, over the course of the following paragraphs, with extensive reference to FIG. 7 (for the method operation blocks) and FIG. 8 (for the software blocks).

Processing begins at operation S752, where instructions broadcast module (“mod”) 852 broadcasts scent instructions to a broadcast transmitter. Broadcast transmitter 612 (see FIG. 6) broadcasts signals to scent capable devices, such as enabled device sub-system 602. In this example, the broadcast transmitter receives scent instructions to be broadcast to a scent-enabled device. Alternatively, a scent dispersion sub-system automatically sends the broadcast transmitter instructions to send pre-defined signals in certain conditions. For example, when an enabled device is proximate a pre-defined location during pre-defined weather conditions, the enabled device causes the broadcast transmitter to send scent instructions.

In this example, a scent-enabled device sends its location to a scent sending service that maintains and/or acquires the services of the transmitter. When the scent-enabled device is within a pre-defined geographic area according to the scent request, the scent sending service transmits the scent instructions to the scent-enabled device.

Alternatively, the broadcast transmitter is linked to a specific location and broadcasts to a pre-determined distance radius.

Some embodiments of the present invention are directed to scent instructions that serve as instructions for scent-emitting devices, also referred to herein as scent-enabled devices or simply enabled devices, to produce a certain scent. The scent may be identical to a natural scent produced by an object or a manufactured scent that either simulates a natural scent or is a scent designed to evoke a particular reaction. For example, scent instructions received from a transmitter direct an enabled device to produce the scent of peppermint. In this example, the scent instructions convey what type of scent is to be produced by a device. Alternatively, a pre-determined set of scents are enabled on the device and are assigned an identification number. The transmitter, in that case, transmits instructions identifying a particular scent by its identification number. Additionally, the scent instructions may include a duration of time to produce the scent.

According to some embodiments of the present invention, the scent instructions include additional information including: (i) a description of the scent; (ii) the address of a specified location for dispensing the scent; and/or (iii) a set of distances, or thresholds, for dispersing a corresponding intensity of scent. After processing the scent, instructions are transmitted by the scent dispersion sub-system via the network to an enabled device, such as mobile phone 606. Regardless of how the specific scent is communicated to the enabled device, in this example, the scent instructions are broadcasted by the transmitter to within a two-mile radius of specified geographic location and the intensity of the scent increases according to half-mile thresholds.

Processing proceeds to operation S754, where instructions receipt mod 854 receives scent instructions on a user's device. Capable devices receive scent instructions from a broadcast transmitter. A capable device may be any device that can detect and send locations, receive instructions, and release or produce a scent. For example, enabled device sub-system 602 (FIG. 6) is capable of releasing a scent via scent generator 714 when scent instructions are received by scent instruction mod 611 operated by transmitter 612. Devices including mobile phone 606, wearable device 604, and wearable device 610 and additional examples of enabled devices that are enabled to produce scent by similar mechanisms as shown for enabled device 602. Some embodiments of the present invention transmit instructions to numerous enabled device over a network, such as network 614.

Additionally, information pertaining to scent instructions, such as a description of the scent and the address of a specified location, may be broadcast to enabled devices of users. Devices may use global positioning systems (GPS), such as GPS 722 (FIG. 6) to detect their location. Based on their detected location, the enabled device may receive scent instructions if it is within a pre-determined distance from a specific location. Alternatively, each enabled device within a particular range receive the instructions, but only those devices within the pre-determined distance or those that arrive within the pre-determined distance during a specified duration of time produce the instructed scent.

If multiple broadcast transmitters broadcast different scents instructions to an enabled device, an algorithm is used to determine which scent instruction is acted upon by the device. Some embodiments of the present invention record user preferences to support a determination as to which scent instruction to follow. Alternatively, user history influences the elected scent instruction.

Processing proceeds to operation S756, where distance determination mod 856 determines distance to specified location based on the address provided in the scent instructions. Scent dispersion program 800 may use GPS to determine the distance and travel path between a user and a specified location. The specified location need not be where the broadcast transmitter is located. The specified location may be any geo-positioned location. Further, the specified location may be associated with a mobile object, such as a food truck, so subsequent scent instructions would provide a latest location of the mobile object as the address of the specified location.

Scent dispersion program 800 may use the location of the user to determine necessary scent output by the scent generator from the enabled device. The travel path may be determined using standard GPS methods for a user traveling in that area. The travel path can be viewed by a user on any networked device such as a mobile phone or a wearable display.

Processing proceeds to operation S758, where scent release mod 858 releases scent from an enabled device. Based on the scent instructions received, enabled devices release a pre-determined amount of scent to the user. The amount of scent released is commensurate with the distance between the user and the specified location. The broadcasting radius may also be factored into the amount of strength output. The strength and the amount of scent that is released is determined based on algorithms that consider, for example, location conditions/weather, and user preferences. For example, certain locations may be windy or have a heavy background odor, so more scent output is needed in order for a user to smell the scent.

Some embodiments of the present invention provide for a single amount of scent having varying intensities depending on the type of scent being released. Accordingly, when a relatively small amount of scent has a very intense, or strong, smell, the amount of scent released would be less than for a weaker scent type. An amount of scent is relative to the type of scent and the form of the scent to be released. Regardless of the type and form, the amount is a corresponding measure of the scent being released.

Because of varying senses of smell among individuals and odor sensitivities, the level of scent output may vary from user to user. Some embodiments of the present invention provide for scent production control based on individual preferences of a user. User preferences may be stored in database 720.

Processing proceeds to operation S760, where scent output determination mod 860 determines whether to increase or decrease of scent output based on user's location. Based on the location of the enabled device, the strength and amount of scent output varies. The closer the user is to the specified location, the stronger and greater amount the scent becomes. Conversely, the farther away the enabled device is to the specified location, the weaker and lesser amount the scent becomes. In some embodiments of the present invention, a baseline scent strength is assigned to the enable device and from that baseline strength increases or decreases according to distance from the specified object. For example, as the user walks toward the specified location, more scent is released via a wearable device. But if the user turns away from the specified location, the scent weakens and is less prevalent in output. A user may use the strength and amount of scent to follow to get to a specified location. The strength and amount of scent released is higher for devices in closer proximity to the location than those that are farther away, creating a gradient of scent strength strongest near the central location and growing weaker as the distance from this central point increases.

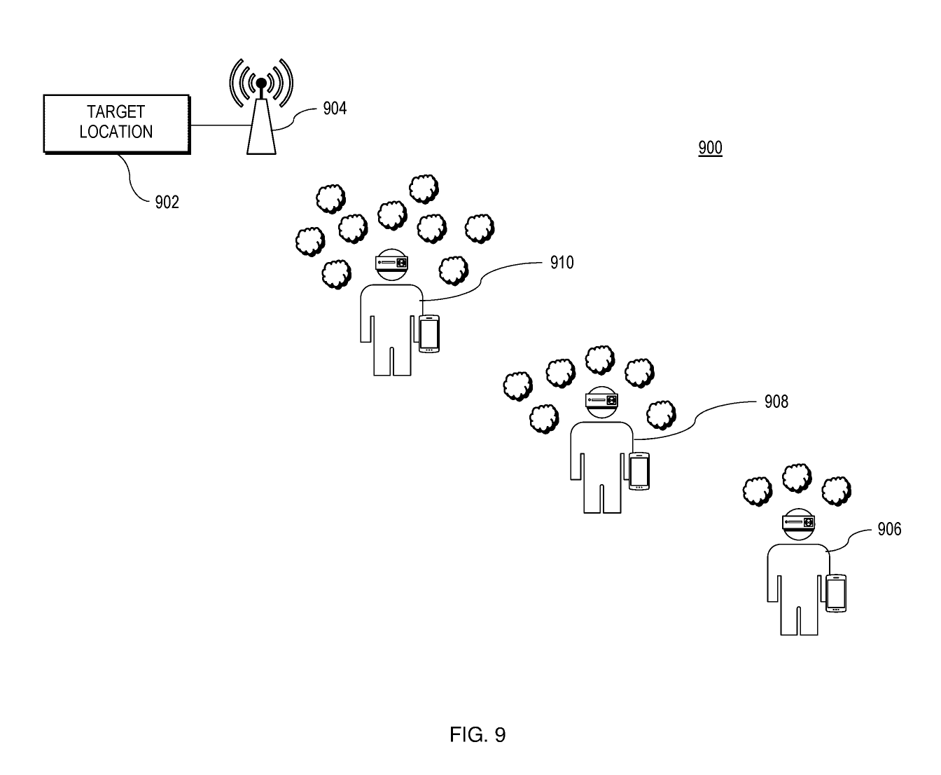

FIG. 9 is an example diagram 900 of users at varying distances from a broadcast transmitter. Specified location 902 is a location that users are near. Specified location 902 may be advertising or otherwise drawing people toward it. Broadcast transmitter 904, interconnected with specified location 902, transmits scent instructions to users wearing wearable odor releasing devices. The scent instructions are designed to lead users to specified location 902. Broadcast transmitter 604 also transmits information and GPS coordinates to users' mobile devices. Users 906, 908, and 910 are users wearing wearable odor releasing devices on their heads and each user is carrying a mobile phone, which may also be enabled to release scents. User 910 is closest to specified location 902, user 908 is farther away from specified location 902, and user 906 is even farther away from specified location 902. The scent clouds above the user's heads represent the amount of scent that has been released to the user from the user's wearable odor releasing devices. As seen in diagram 900, users 906, 908, and 910 have varying amounts of scent released. As a user is closer to specified location 902, more scent is released, and thus more scent clouds are shown about user 910 versus users 908 and 906. Similarly, user 908 is closer to specified location 902, so more scent clouds appear over user 908 compared to user 906.

Some embodiments of the present invention may include one, or more, of the following features, characteristics, and/or advantages: (i) people not wearing a device can have a limited ability to detect scent from residual second hand odors emitted from odor emitting devices other people are wearing; (ii) people not wearing a device can determine the direction of the virtual source of the scent similar to when an odor naturally emanates from a physical source; (iii) scents can be emanated much farther than from a physical object; (iv) scents can be more controlled or manufactured for strength and amount than scent from a physical object; (v) scents me be less subject to weather variables, such as wind or rain; (vi) scent dispersion may be controlled to account for differences in local terrain; and (vii) scents can be used for crowd control, advertisement, or entertainment purposes.

Some embodiments of the present invention create a moving specified location that can be virtually moved around a geographic location. A group of users may be wearing wearable devices that can release scents. A broadcast transmitter may release scent instructions to all wearable devices within a geo-location. Scent instructions may contain GPS coordinates of the virtual specified location, the scent of the specified location, and the size of the specified location. The specified location is the location where the scent released from wearable devices is strongest and most prevalent. Some embodiments may perform the following process: (i) each wearable device determines the amount of scent to be released based on the distance from the GPS coordinates of the specified location, the specified location's size, and possibly other parameters and (ii) each wearable device determines the amount of scent that is released based upon movement of the user wearing the wearable device and/or any broadcast changes in the location or size of the specified location. The specified location can be made to travel, virtually to any geographic location based on the discretion of instructions sent from a broadcast transmitter. As the specified location is moved away from a user, the scent emitting from the user's wearable device becomes weaker. If the user follows the specified location and gets closer to the new GPS coordinates of the specified location, the scent emitting from the user's wearable device becomes stronger. The specified location can, therefore, lead users to multiple locations if the user follows the scent emitted from wearable devices receiving broadcasted scent instructions.

### IV. Definitions

“Present invention” does not create an absolute indication and/or implication that the described subject matter is covered by the initial set of claims, as filed, by any as-amended set of claims drafted during prosecution, and/or by the final set of claims allowed through patent prosecution and included in the issued patent. The term “present invention” is used to assist in indicating a portion or multiple portions of the disclosure that might possibly include an advancement or multiple advancements over the state of the art. This understanding of the term “present invention” and the indications and/or implications thereof are tentative and provisional and are subject to change during the course of patent prosecution as relevant information is developed and as the claims may be amended.

“Embodiment,” see the definition for “present invention.”

“And/or” is the inclusive disjunction, also known as the logical disjunction and commonly known as the “inclusive or.” For example, the phrase “A, B, and/or C,” means that at least one of A or B or C is true; and “A, B, and/or C” is only false if each of A and B and C is false.

A “set of” items means there exists one or more items; there must exist at least one item, but there can also be two, three, or more items. A “subset of” items means there exists one or more items within a grouping of items that contain a common characteristic.

A “plurality of” items means there exists at more than one item; there must exist at least two items, but there can also be three, four, or more items.

“Includes” and any variants (e.g., including, include, etc.) means, unless explicitly noted otherwise, “includes, but is not necessarily limited to.”

A “user” or a “subscriber” includes, but is not necessarily limited to: (i) a single individual human; (ii) an artificial intelligence entity with sufficient intelligence to act in the place of a single individual human or more than one human; (iii) a business entity for which actions are being taken by a single individual human or more than one human; and/or (iv) a combination of any one or more related “users” or “subscribers” acting as a single “user” or “subscriber.”

The terms “receive,” “provide,” “send,” “input,” “output,” and “report” should not be taken to indicate or imply, unless otherwise explicitly specified: (i) any particular degree of directness with respect to the relationship between an object and a subject; and/or (ii) a presence or absence of a set of intermediate components, intermediate actions, and/or things interposed between an object and a subject.

A “module” is any set of hardware, firmware, and/or software that operatively works to do a function, without regard to whether the module is: (i) in a single local proximity; (ii) distributed over a wide area; (iii) in a single proximity within a larger piece of software code; (iv) located within a single piece of software code; (v) located in a single storage device, memory, or medium; (vi) mechanically connected; (vii) electrically connected; and/or (viii) connected in data communication. A “sub-module” is a “module” within a “module.”

A “computer” is any device with significant data processing and/or machine-readable instruction reading capabilities including, but not necessarily limited to: desktop computers; mainframe computers; laptop computers; field-programmable gate array (FPGA) based devices; smart phones; personal digital assistants (PDAs); body-mounted or inserted computers; embedded device style computers; and/or application-specific integrated circuit (ASIC) based devices.

An “intensity” of a scent is an amount of scent adjusted for distance, wind, humidity, and/or other factors.

What is claimed is:

1. A computer-implemented method for distance-based scent emission, the method including:
transmitting, by a scent-generating device, first location information;
receiving scent instructions based on the first location information, the scent instructions including an algorithm for emission of a specified scent; and
determining a first scent emission response according to the scent instructions;
wherein:
the first location information includes a device location at a time of the transmitting action.

2. The computer-implemented method of claim 1, wherein the device location is a set of global positioning system coordinates corresponding to a physical location of the scent-generating device.

3. The computer-implemented method of claim 1, wherein the scent instructions further include: the specified scent, a target location, and a distance gradient including a maximum response distance.

4. The computer-implemented method of claim 3, wherein determining the first scent emission response includes:
determining that a calculated distance between the device location and the target location is less than the maximum response distance; and
determining a first intensity of the specified scent to release based on the calculated distance.

5. The computer-implemented method of claim 1, wherein the scent instructions include a set of components making up the specified scent.

6. The computer-implemented method of claim 1, further comprising:
registering, by the scent-generating device, with a scent distribution network for receiving the scent instructions.

7. The computer-implemented method of claim 1, further comprising:
responsive to a scent emission condition being met, determining, by the scent-generating device, a second scent emission response.

8. The computer-implemented method of claim 7, wherein the scent emission condition is movement of the scent-generating device a particular distance from the device location.

9. A method comprising:
determining a device location of a first scent enabled device; and
responsive to the device location being within a pre-defined distance from a target location, causing the first scent enabled device to generate a scent having a first characteristic being variable according to a distance between the device location and the target location
wherein:
the first characteristic is a measure of scent generated; and
at least the determining and the causing the first scent enabled device to generate a scent steps are performed by computer software running on computer hardware.

10. The method of claim 9, wherein the scent is selected from a set of pre-defined scents that the first scent enabled device is enabled to generate.

11. The method of claim 9, further comprising:
causing the first scent enabled device to stop generating the scent based on a stop condition;
wherein the stop condition is a duration of scent generation.

12. The method of claim 9, further comprising:
receiving a request to cause an available scent enabled device to generate the scent; and
identifying the first scent enabled device within a set of available scent enabled devices based on the request;
wherein:
the request includes a scent type, a target location, the pre-defined distance from the target location, and a duration of scent generation.

13. The method of claim 12, further comprising:
determining the first scent enabled device is outside the pre-defined distance from the target location; and
responsive to the first scent enabled device being outside the pre-defined distance, causing the first scent enabled device to stop generating the scent.

14. The method of claim 9, further comprising:
monitoring a second scent enabled device for location information;
determining, based on the location information, the second scent enabled device is within the pre-defined distance from the target location; and
causing the second scent enabled device to generate a scent having a second characteristic being variable according to an actual distance between the second scent enabled device and the target location;
wherein:
the second characteristic is a measure of scent generated.

15. A computer-implemented method comprising:
receiving, by a scent-enabled device, a first scent instruction including a specified scent and a target location;
determining a first distance from the target location based on a first location of the scent-enabled device;
determining a first characteristic of the specified scent based on the first distance; and
releasing the specified scent according to the determined first characteristic.

16. The computer-implemented method of claim 15, wherein the specified scent is a set of scent components, each available to the scent-enabled device.

17. The computer-implemented method of claim 15, wherein the target location is associated with a mobile object.

18. The computer-implemented method of claim 15, wherein the first characteristic is an intensity of scent to be released.

19. The computer-implemented method of claim 18, wherein the intensity of scent to be released is adjusted according to a user preference identifying a baseline intensity of scent.

20. The computer-implemented method of claim 15, wherein the first scent instructions further includes pre-defined intensities of scent to be released according to a set of distances representing thresholds for release specified intensities of scent.
